(19) United States
(12) Patent Application Publication
(10) Pub. No.: US 2023/0301806 A1
(43) Pub. Date: Sep. 28, 2023

(54) Implant For Treating Aneurysms

(71) Applicant: phenox GmbH, Bochum (DE)

(72) Inventors: Hans HENKES, Stuttgart (DE); Hermann MONSTADT, Bochum (DE); Ralf HANNES, Dortmund (DE); Volker TRÖSKEN, Witten (DE); Stefan ROLLA, Bochum (DE)

(73) Assignee: phenox GmbH, Bochum (DE)

(21) Appl. No.: 18/009,327

(22) PCT Filed: Jun. 14, 2021
(86) PCT No.: PCT/EP2021/065958

§ 371 Date: Dec. 8, 2022

(30) Foreign Application Priority Data
Jun. 12, 2020 (DE) 10 2020 115 605.7

(51) Int. Cl. A61F 2/856; A61F 2/90

(52) U.S. Cl. CPC A61F 2/856; A61F 2/90

(57) ABSTRACT

An implant (6, 18) for influencing the flow of blood in the area of bifurcation aneurysms with the implant (6, 18) having a first (1, 14) and a second tubular braided section (2, 15) each having an opening (4, 5, 16, 17) in the wall, wherein the openings (4, 5, 16, 17) in the walls being produced in such a way that the wires (3) forming the braided sections (1, 2, 14, 15) are radially displaced at the positions of the openings (4, 5, 16, 17), and the first braided section (1, 14) is guided through the opening (5, 17) in the wall of the second braided section (2, 15) such that the opening (4, 16) in the wall of the first braided section (1, 14) points to the opening (5, 17) in the wall of the second braided section (2, 15).

The invention relates to an implant for influencing the flow of blood in the area of aneurysms that are localized at vessel branches. Aneurysms of this type are also known as bifurcation aneurysms.

Aneurysms are usually saclike or fusiform dilatations of the vessel wall and occur primarily in structurally weakened areas of the vessel wall due to the constant pressure of blood. Accordingly, the inner vessel walls of an aneurysm are thus sensitive and susceptible to injury. Rupture of an aneurysm usually leads to significant health impairment, and in the case of cerebral aneurysms, to neurological deficits and even fatalities of patients.

Aside from surgical interventions, in which, for example, the aneurysm is clamped by means of a clip, endovascular methods for the treatment of aneurysms are known in particular, with two approaches being primarily pursued. One option includes filling the aneurysm with occlusion means, especially using so-called coils (platinum spirals) for this. Coils facilitate the formation of thrombi and thus ensure occlusion of the aneurysm. On the other hand, it is known to close off the access to the aneurysm, for example the neck of a berry aneurysm, from the blood vessel side making use of stent-like implants and in this manner disconnect it from the blood flow. Both methods serve to reduce the blood flow into the aneurysm and in this way alleviate, ideally even eliminating the pressure acting on the aneurysm and thus reducing the risk of an aneurysm rupture. When filling an aneurysm with coils it may happen that the filling of the aneurysm is inadequate, allowing blood flow into the aneurysm and in this way cause the pressure acting on its inner wall to continue. The risk of steady dilation of the aneurysm and eventually its rupture persists, albeit in an attenuated form. Moreover, this treatment method is only suitable for aneurysms having a relatively narrow neck - so-called berry aneurysms - as otherwise there is the risk that coils protrude from a wide aneurysm neck into the blood vessel where they produce clots, which may lead to occlusions in the vessel. In the worst case, a coil is completely washed out of the aneurysm and causes vessels to be occluded elsewhere. To keep the coils in place in the aneurysm sac, the aneurysm neck is often additionally covered with a special stent.

Another intravascular treatment approach focusses on so-called flow diverters. These implants are similar in appearance to stents that are used for the treatment of stenoses. However, since the purpose of the flow diverters is not to keep a vessel open, but to obstruct access to the aneurysm on the blood vessel side, their mesh width is very narrow; alternatively, implants of this kind are coated with a membrane. A disadvantage of these implants is the risk that outgoing side branches in the immediate vicinity of the aneurysm to be treated are sometimes also covered and thus closed off in the medium or long term.

Vessel branches, in particular vessel bifurcations are a quite frequently occurring phenomenon. In the event of a weak vessel wall, the blood stream flowing through an artery and acting on the front wall in a bifurcation quickly causes a protuberance or bulge which is prone to rapidly dilate further. More often than not, such bifurcation aneurysms have a wide neck which makes therapy difficult to be performed with occlusion coils only.

Vascular implants that are suitable to bring about such a “barring” of the aneurysm entrance in the area of a vascular branching have been disclosed, for example, in the international patent applications WO 2012/113554 A1 or WO 2014/029835 A1. The aneurysm can then be rendered non-hazardous as a result of occlusion coils inserted after the implant has been placed in position. It is also possible that the implant itself separates the aneurysm sufficiently from the blood flow. For this purpose, for example, the implant may have a membrane that is placed in the area of the aneurysm neck or in front of the aneurysm neck. If considered useful or expedient, the blood flow to the aneurysm can also be reduced with filaments alone, typically wires of small diameters, to such an extent that the additional introduction of occlusion coils or other occlusion means into the aneurysm can be dispensed with.

From WO 2018/208662 A1 a flow diverter system for the treatment of bifurcation aneurysms is known, by means of which a stent-like flow diverter with a lateral opening is first inserted into the blood vessel. Subsequently, another stent-like flow diverter provided with lateral opening is passed through the opening of the first flow diverter in such a way that a Y-shape is formed overall, with the branching in the Y being located in front of the bifurcation aneurysm. In this approach, the aneurysm should be cut off from the blood flow; however, the blood flow into the branching blood vessels should remain largely unaffected.

A disadvantage of this prior art approach is that the two elements of the final flow diverter have to be inserted one after the other. This requires initially that the first flow diverter must be placed correctly before the second flow diverter has to be accurately positioned within the vasculature both relative to the aneurysm as well as relative to the first flow diverter. Due to fact that for the treatment of bifurcation aneurysms in the intracranial region implants are as a rule inserted via the femoral artery, the treating physician must therefore have and perform very precise control over considerable distances, with the additional impediment that visualization of the implants, in particular the precise alignment of the openings inside the body, is difficult.

Based on the prior art described hereinbefore, it is thus the objective to provide an implant that enables the treatment of bifurcation aneurysms, but that can be brought to the target position as a unit.

As proposed by the present invention this objective is achieved by an implant for influencing the flow of blood in the area of aneurysms which are localized at vessel branchings, with the implant being provided in an expanded state in which it is implanted in the blood vessel and in a contracted state in which it is movable through the blood vessel, with the implant having a first and a second braided section which are tubular in the expanded state and the walls of which are constructed from individual wires interwoven with one another, the first and second braided sections each having an opening in the wall, with at least the size of the opening in the second braided section being sufficient for the passage of the first braided section therethrough, the openings in the walls being produced in such a way that the wires forming the braided sections are radially displaced at the positions of the openings, and the first braided section is guided through the opening in the wall of the second braided section such that the opening in the wall of the first braided section points to the opening in the wall of the second braided section.

Accordingly, the implant is not assembled inside the body at a position immediately adjacent to the aneurysm, but before it is introduced into the vascular system. This will considerably facilitate the correct placement of the implant.

It is also significant that the openings in the braided sections are created at the respective positions by displacing the wires forming the braided section laterally, i.e. radially, over the circumference of the braided section. Therefore, rather than creating an opening by cutting out wires, the wires themselves are maintained unaffected without introducing a break in the wires. The wires are merely moved or shifted sideways. This is of significance in that it enables the implant in the compressed/contracted state to be reliably maneuvered to the target position. It is to be borne in mind that the advancement/feed of wires that are interrupted is often not easily possible.

Moreover, the design of the implant with wires of uninterrupted configuration permits for the trouble-free expansion of the implant at the target position in the blood vessel. Since, according to the invention, the wires are not interrupted, expansion is unproblematic.

An additional advantage is that no loose wire ends are present in the area of the openings that could injure the adjacent vessel walls or even protrude into the channel of the blood vessel where they could cause obstruction of the blood flow. Preventing injury to the vessel wall is also of particular importance because the implants are placed in the vicinity of a bifurcation aneurysm, a zone already characterized by weakened vessel walls.

In accordance with a first embodiment of the invention, the inventive implant is manufactured by first creating two braided sections, each provided with an opening in the side wall. The first braid section is then passed through the opening in the second braided section and partially pulled through the interior of the second braided section. Alignment takes place in such a way that the two openings of the braided sections are facing each other. In this way, a passage is created in the transition area so that the blood inflow from the parent blood vessel can readily flow into and enter both branching blood vessels.

The number of wires forming the respective braided sections advantageously amounts to between 24 and 96, more preferably to between 36 and 64, by which the numbers apply per braided section. A relatively high number of wires ensures a high surface density, especially in the zone in front of the aneurysm neck, so that the inflow of blood is prevented. On the other hand, the stiffness of the implant increases with the amount of wires provided.

The openings in the walls of the first and/or second braided sections are conveniently arranged approximately in the middle of and within the braided section, with ‘in the middle’ referring to the longitudinal direction. In this way, sufficient lengths of the braided section are available in both directions for placement in the respective arms of the blood vessel. This arrangement results in one arm of the Y-shaped implant to be at least partially double-layered, that is, two braided sections are located one on top of the other.

At least one, but preferably both openings have a size such that the other braided section can be passed through the opening. Expediently, both openings are created to be of the same size in order to ensure the blood flow is as uniform as possible and additional inflow resistance is avoided.

Another useful further embodiment provides that in the expanded state in the arm of the Y-shaped implant with superimposed braided sections, these sections overlap. In other words, one braided section in this arm is designed to be longer than the other. Preferably, the overlap is arranged in such a way that the inner braided section projects beyond the outer braided section. Thus, the end of this arm is only single-layered, and in the single-layered section, the inner braided section is as well in contact with the vessel wall. In this way, the inflow resistance for the blood is reduced and the risk of thromboembolic effects lowered.

The first and second braided sections may be joined together at the outer end of the segment in which the two braided sections jointly extend. This is a second embodiment of the implant proposed by the invention, said implant being typically manufactured differently, namely by first providing a braided structure which is tubular in the expanded state and the wall of which is composed of individual wires braided together, wherein the braided structure having a first and a second braided section which are longitudinally adjacent and arranged one after the other, wherein both braided sections each having an opening in the wall, with at least the size of the opening in the second braided section being sufficient for the passage of the braided structure therethrough, wherein the two openings being located on opposite sides of the braided structure and the first braided section being turned over inwardly, passing through the interior of the second braided section and extending through the opening in the second braided section from the inside to the outside in such a manner that the opening in the wall of the first braided section faces the opening in the wall of the second braided section.

Also in accordance with this embodiment, a Y-shaped implant is provided, which is already completely available outside the body and allows the aneurysm to be disconnected from the blood flow, while the circulation through the blood vessels themselves remains unimpeded, but in which, unlike in the first embodiment, there are not two separate braided sections that are brought together, but one braided structure is provided comprising two braided sections.

The first braided section is first pulled through its own interior and then at least partially through the interior of the second braided section, in a sense analogous to turning a sock inside out. The result is an arm of the Y-shaped implant that is double-layered, i.e. in which two braided sections lie on top of each other. As a result, an implant similar to that of the first embodiment is obtained, but the two braided sections at the end of the double-layered arm are connected, because the implant is created from a single braided structure as explained. The double layer configuration increases the radial forces and can thus improve anchoring the implant in the blood vessel. Due to the fact that the two braided sections are connected to each other at the outer end of the segment in which the two braided sections jointly extend, i.e. are double-layered, there are no free wire ends at this point, which is advantageous in that the risk of injury to the vessel wall is reduced. Moreover, wire ends are prevented from protruding into the vessel lumen (so-called fishmouth effect) and obstructing the flow of blood.

For the standard placement of the implant, the double-layered arm of the Y-shaped implant is located in the parent blood vessel from which the two branching blood vessels originate.

The braided structure, which in the second embodiment has two braided sections arranged one after the other in the longitudinal direction, may be constructed, for example, from 32 to 48 wires, taking into account, compared with the first embodiment, that the wires continue between the braided sections, whereas in the first embodiment each braided section is constructed individually from a specific number of wires.

Same as with the first embodiment, the openings in the walls of the first and/or second braided sections are each expediently arranged approximately in the middle within the braided section, with ‘in the middle’ referring to the longitudinal direction. In this way, sufficient lengths of the braided section remain in both directions for placement in the respective arms of the blood vessel. In this embodiment as well, at least one, but preferably both openings have a size such that the other braided section can be passed through the opening.

Thus, in both embodiments, of the 3 arms of the Y-shaped implant, one is at least partially double-layered, and the other two are single-layered. In case of need, the double-layered arm generates higher radial forces and thus enables secure fixation of the implant in the blood vessel system, especially in the parent blood vessel from which the other two blood vessels branch off.

Normally, i.e. with a standard implant placement procedure, the aneurysm to be treated is located at the point of bifurcation between the first and second braided sections after the implant has been assembled. Since the braided sections are located immediately adjacent to the aneurysm neck, the aneurysm is largely cut off from blood flow. Nevertheless, the flow of blood into the actual blood vessels is ensured by the openings in the braided sections facing each other or towards each other.

It is also possible to position the implant in such a way that another branch point of the Y-shaped implant is placed in front of the aneurysm neck. In this case, only part of the first or second braided section extends through the parent blood vessel, while the area where the two braided sections are superimposed is inserted into one of the two branching blood vessels. The other branching blood vessel in this case contains the third arm of the Y-shaped implant, said arm being constructed from a single layer of the other braided section. Such placement may have advantages in that the coverage rate immediately in front of the aneurysm neck may be greater because there is a higher braid density at this location.

To improve the fixation of the two braided sections to each other, they can be attached to each other in the area of their openings. This can be done in particular by sewing, especially with the aid of wires, by forming loops around the adjacent wires of the two braided sections, by gluing or the like. In this manner, an implant is produced in which the two braided sections have a firm connection with each other.

The implant proposed by the invention has a braided structure, i.e. it consists of wires that are braided together by passing them over and under each other. Such a structure is particularly suitable to expand and adapt to the vessel walls after it has been released in the blood vessel.

The implant to a great extent or entirely isolates the bifurcation aneurysm from the flow of blood because at least some portion of the wires comes to rest in front of the aneurysm neck. In contrast, blood flow through the parent blood vessel into the branching blood vessels is virtually unaffected. As a result, the aneurysm atrophies due to the lack of blood movement in the aneurysm causing a thrombus to form and obstruct the aneurysm.

The terms tubular braided structure or tubular braided section shall be understood to define a structure in which the wires form the wall of the tube. It should therefore be considered a round braiding. Preferably, the braided sections/structure have a circular cross-section when viewed from the proximal or distal end. However, the braid may also have a shape other than circular, for example an oval cross section may be provided.

Basically, the braiding may be plaited in any known way. It may have a one-plaited and/or multi-plaited structure. Especially when used in a narrowly plaited configuration a dense braiding will cause the individual wires to be highly stressed. However, while a multi-plaited design is conducive to removing stresses from the braid, a too highly plaited configuration on the other hand will cause the bond in the braid to deteriorate. The plaiting method indicates how many times a given wire passes crossing wires on the same side of such wires before it changes sides and subsequently passes on the other side of a corresponding number of crossing wires. In case of a two-plaited arrangement a wire, for example, passes in succession over two crossing wires and then in succession along the underside of two crossing wires. In a one-plaited structure the wires are arranged alternately one above the other and one below the other.

In particular, also multi-ply wires may be employed. The plying indicates the number of joined, parallelly arranged individual wires. Single or multiple plying may be provided with one or several individual wires extending in parallel. Since during the braid manufacturing process wires are introduced into the process from bobbins, one or several individual wires are fed from the respective bobbin simultaneously to the mandrel on which the braiding is produced. Each wire may consist of a single wire or of strands comprising several individual wires joined and preferably twisted together.

A plying of two or an even higher plying configuration results in a higher surface density of the braiding and at the same time reduces the longitudinal expansion when the braiding is compressed. Such a higher surface density, however, causes flexibility to diminish, also through increased friction and tension. This may be counteracted by making use of a more highly plaited arrangement, i.e. a two-plaited or higher-plaited structure will result in higher flexibility.

Different forms of filaments can be used as wires. These may have a round, oval or even angular cross-section, in particular a rectangular, square or trapezoidal cross-section, and in the case of an angular cross-section the edges can be rounded. Flat wires in the form of thin strips can be used as well. The individual wires can also be made up of several individual filaments that are twisted together or extending in parallel. The wires can be solid or hollow inside. Additionally, the wires may be subjected to electropolishing to make them smoother and even rounder and thus render them less traumatic. This also reduces the risk of germs or other impurities adhering.

Preferably, the wires are made of metal, but in principle the use of wires made of other materials, for example plastics or polymer materials, is also conceivable. Filaments of this type are also understood as wires according to the invention.

To ensure that the implant, when released in the blood vessel, for example from or out of a catheter, automatically expands and adapts to the inner walls of the blood vessels, it is preferred to make the wires at least partially from a material having shape memory properties. Nickel-titanium alloys, for example nitinol, or ternary nickel-titanium-chromium alloys or nickel-titanium-copper alloys are particularly preferred in this context. However, other shape memory materials, for example other alloys or even shape memory polymers, are also conceivable. Materials having shape memory properties allow an implant to be imprinted with a secondary structure that it will automatically strive to adopt as soon as it is no longer hindered from expanding.

It is also possible to use so-called DFT® (drawn filled tubing) wires, i.e. wires in which the core of the wire consists of a different material than the sheath surrounding the core. It is particularly expedient to use wires having a core of a radiopaque material and a sheath of a material with shape memory properties. The radiopaque material may, for example, be platinum, a platinum-iridium alloy, or tantalum, and the material having shape memory properties is preferably a nickel-titanium alloy, as mentioned above. DFT® wires of this type are offered by Fort Wayne Metals, for example.

Respective wires combine the advantageous characteristics of two materials. The sheath having shape memory characteristics will ensure that the implant is allowed to expand and adapt to the vessel walls, whereas the radiopaque material ensures that the implant is visible on radiographs and can thus be observed by the attending physician and positioned as needed.

The distal and proximal ends of the wires are preferably configured with a view to precluding injury to the vessel walls. For example, wires can be rounded at their ends and thus made atraumatic. Appropriate forming can be done by remelting with the help of a laser. It is also possible to join one or more wires at each end and thus create appropriate atraumatic terminations. It is, in particular, expedient to avoid pointed wire ends.

Advantageous implant coverage rates, in particular also at the branching point where the aneurysm neck is covered by implantation, are 45 to 75%, preferably 35 to 65%, for the implant in the expanded state.

Unless the context indicates otherwise, the term expanded state within the meaning of the invention is understood to denote a state which the implant assumes when it is not subject to any external constraints. Depending on the diameter of the blood vessels in which the implant is implanted, the expanded state in the vasculature may differ from the expanded state existing in the absence of external constraints because the implant may not be able to reach its completely expanded state. In the completely expanded state, the braided sections advantageously have an outer diameter ranging between 1.5 mm and 7 mm, said diameter can be adapted to the respective target site in the blood vessel system. The overall length of the implant in the expanded state is usually between 5 mm and 100 mm, and in particular between 10 and 50 mm when the implant is placed such that the two single-layer arms of the Y-shaped implant are parallel and extend in the same direction as the double-layer arm. The wires used for the implant may, for example, have a diameter or thickness ranging between 20 and 60 µm.

On the other hand, the implant may also be in a contracted or compressed state, the terms being used synonymously in the context of the present invention in the sense that the implant or a braided section/the braided structure has a significantly smaller radial extent in the contracted/compressed state than in the expanded state. A contracted/compressed state is assumed, for example, when the implant is advanced to the target position by a catheter. It is also possible to mount the implant on the outside of a catheter, tube or the like, in which case the implant is also maintained in a less radially enlarged state as compared to its expanded state.

With respect to the implant placement process, the terms “proximal” and “distal” are to be understood such that they refer to parts of the implant that point towards the attending physician (proximal), or, as the case may be, to parts that point away from the attending physician (distal). Typically, the implant is thus moved forward in distal direction by or with the aid of a catheter. The term “axial” refers to the longitudinal axis of the implant extending from proximal to distal while the term “radial” denotes levels/planes extending perpendicular thereto.

To further improve the occlusion of the aneurysm, in addition to isolating the aneurysm from the flow of blood by the implant, occlusion agents may also be introduced into the aneurysm, for example coils as they are known in the prior art. Also possible is the insertion of viscous embolic agents such as onyx.

The implant proposed by the invention is usually provided with radiopaque marker elements facilitating visualization and positioning accuracy at the placement site. Such marker elements can e.g. be provided in the form of wire coils, as sleeves and as slotted tube sections that are secured to the implant. For said marker elements, in particular platinum and platinum alloy materials are suitable, for example an alloy of platinum and iridium, as it is frequently used according to the state of the art for marking purposes and as material for occlusion coils. Other usable radiopaque metals are tantalum, gold, and tungsten. Another option is to fill the wires with a radiopaque material, as mentioned hereinbefore. It is also possible to provide the implant, in particular the wires, with a coating consisting of a radiopaque material, for example applying a gold coating. This coating can, for example, have a thickness of 1 to 6 µm. The radiopaque material coating need not be applied to the entire implant. Nevertheless, even when applying a radiopaque coating it is considered useful to arrange one or several radiopaque markers on the implant, in particular at the distal end of the implant.

The implant may also be provided with membranes that at least partially cover the braided sections, with a membrane may be used that extends over larger areas of the braided sections or a plurality of small membranes can be provided. Such a membrane is particularly useful at the neck of the aneurysm to cut off the aneurysm from the blood flow, i.e., at the branching point in distal direction.

Covering by a membrane shall be understood to mean any type of covering, that is, the membrane may be applied to the outside of the braided sections, attached to the inside of the braided sections, or the wires of the braided sections may be embedded in the membrane.

If one or more membranes are employed, it is also possible to introduce into the membranes substances that are opaque to radiation. These may be radiopaque particles as they are customarily employed as contrast medium for radiotechnological purposes. Such radiopaque substances are, for example, heavy metal salts such as barium sulfate or iodine compounds. A radiopaque membrane proves beneficial during placement of the implant and for localization purposes and may be used either additionally to or instead of marker elements.

Membranes may also be designed to have an antithrombogenic effect or an effect that promotes endothelial formation. Such an effect is particularly desirable where the implant is adjacent to normal vessel walls because blood flow through the vessels should not be impaired and, moreover, good anchorage of the implant in the vascular system should be achieved. Membranes can possess the desired characteristics by themselves through an appropriate choice of material, but they can also be provided with coatings that produce these effects.

Within the meaning of the present invention, a membrane is a thin structure having a planar surface, regardless of whether said structure is permeable, impermeable or partially permeable to liquids. However, to accomplish the objective of the aneurysm treatment, membranes are preferred that are completely or at least substantially impermeable to fluids such as blood. In addition, a membrane may also be provided with pores, particularly in the region of the aneurysm neck, through which occlusion agents can be introduced into the aneurysm. Another option is to have the membrane designed in such a way that it can be pierced with a microcatheter for the introduction of occlusion agents or even with the occlusion agents themselves.

The membranes can be made of polymer fibers or polymer films. Preferably, the membranes are produced by an electrospinning process. In this process, the wires are normally embedded in the membrane which can be achieved by weaving fibers around the wires or braiding them. In electrospinning, fibrils or fibers are separated from a polymer solution and deposited on a substrate by applying an electric current. Said deposition causes the fibrils to agglutinate into a non-woven fabric. Usually, the fibrils have a diameter ranging between 100 and 3000 nm. Membranes created by electrospinning have a very uniform texture. The membrane is tenacious, withstands mechanical stresses, and can be pierced mechanically without an opening so created giving rise to cracks propagating from it. The thickness of the fibrils as well as the degree of porosity can be controlled by selecting process parameters as appropriate. In the context of producing the membrane and with respect to materials suitable for this purpose, special attention is drawn to publications WO 2008/049386 A1, DE 28 06 030 A1, and literature referred to therein.

In lieu of electrospinning, the membranes may also be produced by an immersion or spraying process such as spray coating. With respect to the material used for the membranes, it is important that they are not damaged by the mechanical stresses arising during implant insertion into the blood vascular system. To ensure this, the membranes should have sufficient elasticity.

The membranes can consist of a polymer material such as polytetrafluoroethylene, polyester, polyamides, polyurethanes, polyolefins or polysulfones. Especially preferred are polycarbonate urethanes (PCU). In particular, an integral connection between the membranes and the wires is desirable. Such an integral connection can be achieved by covalent bonds provided between the membranes and the wires. The formation of covalent bonds is promoted by silanization of the wires, i.e. by a chemical bonding of silicon compounds, in particular silane compounds, to at least portions of the wire surface. On surfaces, silicon and silane compounds attach, for example, to hydroxy and carboxy groups. Basically, aside from silanization, other methods of mediating adhesion between the wires and the membranes are also conceivable.

Silane compounds in this context are to be seen as all those compounds which follow the general formula RmSiXn (m, n = 0-4), where R stands for organic radicals, in particular alkyl, alkenyl or aryl groups, and X stands for hydrolyzable groups, in particular OR, OH or halogen with R = alkyl, alkenyl or aryl. In particular, the silane may have the general formula RSiX3. Moreover, relevant compounds having several silicon atoms also count among the silane compounds. In particular, silane derivatives in the form of organosilicon compounds are regarded as silane compounds in this context.

As already mentioned hereinbefore, additional substances promoting endothelial formation may be embedded in or deposited on the membranes. Because aneurysms are due to degenerative diseases of the vascular wall, the promotion of endothelial formation and correction of endothelial dysfunction may yield beneficial effects. This applies especially to the area where the aneurysm is in contact with the flow of blood in the respective blood vessel (parent vessel). Preferably, substances promoting endothelial formation are applied to the outside of the membrane, with outer side being understood here to denote the side of a membrane facing the vessel wall in the implanted state and the inner side being understood to mean the membrane side facing away from the vessel wall. Hyaluronic acid, statins (3-hydroxy-3-methylglutaryl coenzyme A reductase inhibitors), and other polymers may promote endothelial cell colonization. Polysaccharides, especially glycosaminoglycans, which are able to mimic the glycocalyx, are particularly suitable polymers. Another material that can be used is POSS-PCU (polyhedral oligomeric silsesquioxane poly(carbonate-urea) urethane). This material is a nanocomposite that has been described inter alia as a scaffold for artificial organs and as a coating for medical devices, (Tan et al., Crit Rev. Biomed Eng. 2013; 41(6): 495-513). It is also possible to use POSS-PCL (polyhedral oligomeric silsesquioxane poly(caprolactone-urea) urethane). It applies to both POSS-PCU and POSS-PCL that in particular functionalized derivatives of these nanocomposites can also be employed. This is especially true for those derivatives that can be obtained by linking with polyacrylic acid (poly-AA). Since POSS-PCU and/or POSS-PCL nanocomposite polymers are only poorly suited for direct immobilization on the surface of an implant, it has been found advantageous to combine polymers such as polyacrylic acid (poly-AA) with the nanocomposite. This can be achieved, for example, by plasma polymerization of acrylic acid. A poly-AA-g-POSS-PCU surface obtained in this way promotes collagen bonding (especially collagen type 1) and thus endothelial formation (cf. Solouk et al., Mater Sci Eng C Mater Biol Appl. 2015; 46: 400-408). In general, biofunctional or bioactive coatings may be present on the membrane.

The implant provided by the invention is particularly suitable for the treatment of intracranial bifurcation aneurysms, but its use for other types of aneurysms, for example aortic aneurysms or peripheral aneurysms, is also conceivable, in which case the dimensions of the implant are to be adapted as appropriate.

To place the implant in front of the bifurcation aneurysm, an insertion system can be used as described in publication WO 2018/134097 A1. As a further development in this context serves an insertion system which comprises a sleeve with two distal sleeve arms which are each configured to receive a distal implant arm and are connected to one another, wherein the sleeve has a continuous opening zone in the distal direction and the sleeve has a proximal sleeve arm for receiving the proximal implant arm, the sleeve being retractable proximally via the proximal sleeve arm such that the opening zone opens and the distal implant arms each pass through the opening zone and are released in the branching blood vessels, the opening zone being configured such that upon release of the implant the sleeve opens sequentially in proximal direction from the distal ends of the distal sleeve arms. In contrast to the insertion system described in publication WO 2018/134097 A1, such an insertion system differs in that the implant is gradually liberated from distal to proximal and is allowed to expand and adhere to the vessel wall accordingly, which is considered advantageous.

In this respect, the sections of the implant that are placed in the branching blood vessels are referred to as distal implant arms, and the section for placement in the parent blood vessel is referred to as the proximal implant arm.

For this purpose, the opening zone can be designed in different ways. In particular, from the outset, the opening zone may have a continuous slot, facing in the distal direction, but with the edges of the slot at least partially overlapping. This overlap should increase from the two distal ends of the sleeve arms in the proximal direction. In other words, the respective edges of the slots at the distal ends of the sleeve arms do not overlap or at most overlap slightly, but where the sleeve arms meet and are joined together, the two edges of the slot overlap significantly. Accordingly, the least force is required to release the distal implant arms at the distal ends of the sleeve arms, while this force is relatively high where the sleeve arms meet. The slot thus opens first at the distal ends of the sleeve arms and the opening continues in proximal direction so that the implant is released later in the center. This center coincides with the location that is typically placed in front of the aneurysm neck.

Alternatively, the opening zone may have a weakening zone facing in the distal direction, with the force required for opening increasing in this weakening zone from distal to proximal. This zone of weakness can be achieved, for example, via a perforation in which the thickness of the webs between the openings increases from distal to proximal and/or the size of the openings increases from proximal to distal. Another possibility is to provide a thinning of material or even a variation of material in the area of the weakening zone so that the weakening zone at the distal ends of the sleeve arms tears open first when the sleeve is retracted proximally, and the opening of the weakening zone propagates in proximal direction, i.e. to the branching point of the sleeve. The characteristics of the weakness zone thus causes it to tear open as the sleeve is withdrawn from the distal ends of the sleeve arms to the location where the sleeve arms join. This also ensures a sequential release of the distal implant arms, with the more distally arranged areas being released and expanding first and the more proximal areas only slightly later.

Another insertion system that enables the inventive implant to be placed in front of the aneurysm comprises two sleeves, each configured to accommodate a distal implant arm, wherein the two sleeves each have a distal sleeve section with the distal sleeve sections each having an opening zone extending in the longitudinal direction, wherein proximally adjoining each of the distal sleeve sections is a proximal section via which the sleeves can be retracted in the proximal direction so that the opening zones open and allowing each of the distal implant arms to pass through the opening zones and being released in the branching blood vessels. Moreover, a trunk sleeve is provided to accommodate the proximal implant arm, which is placed in the parent blood vessel. For the purpose of releasing the proximal implant arm independently of the sleeves for the distal implant arms, the trunk sleeve can be retracted in the proximal direction, or the trunk sleeve can be retained to also release the proximal implant arm by advancing it in the distal direction. The trunk sleeve must therefore continue in the proximal direction, with the proximal region not necessarily being sleeve-shaped, but can also consist, for example, of a wire or a similar item.

In this insertion system, a sleeve is provided for each of the two distal implant arms which are to be placed in the blood vessels branching off from the parent blood vessel. The sleeve restricts the implant arms and prevents them from expanding radially. At least in the distal region, both sleeves are provided with a longitudinally extending opening zone through which the distal implant arms can pass and be released when the sleeves are retracted in the proximal direction. Alternatively, releasing the implant can be brought about by holding the sleeves in place with the aid of the proximal sections, while advancing the implant with the two distal implant arms in distal direction. The relative movement between the sleeves and the implant is of importance for the release, with the relative movement of the sleeves acting in the proximal direction, whereas the relative movement of the implant acts in the distal direction. In this context, an advancing device can be employed to exert a force on the implant in the proximal direction, either to hold it in place while the sleeves are pulled proximally or to move the implant forward in distal direction.

An opening zone in this case refers to an area of the distal sleeve sections that extends in longitudinal direction from the distal end of the distal sleeve sections proximally, typically to the beginning of the proximal sections. Preferably, the opening zones should point in the distal direction to facilitate the exit of the distal implant arms. The opening zones must reach in proximal direction to such an extent that at least a sufficiently long section of the implant can be accommodated by the sleeves and the implant is safely held by the insertion system during insertion. At the proximal ends of the distal sleeve sections, however, the implant arms protrude from the sleeves, while the sleeves are provided with proximal sections that extend further in the proximal direction.

In particular, the opening zones may consist of longitudinal slots through which the distal implant arms can pass when the sleeves are retracted in the proximal direction. The longitudinally extending edges of the longitudinal slots may abut or overlap to some extent, in each case ensuring that passage of the implant arms remains possible. In this context, longitudinal direction means that the opening zone must extend from the distal end of the distal sleeve sections in the proximal direction, although a course that has a certain radial component is also understood to be “in the longitudinal direction”, for example a helical course.

Instead of arranging for a longitudinal slot, it is also possible to provide another opening zone in which the longitudinal slot is only created during the release process. For example, this may be a weakening zone in which a perforation is provided that opens when the sleeve sections are pulled back, or another option may be used to provide a weakening zone based on material thinning.

It is also considered expedient to provide the opening zone in such a way that the distal implant arms to be released are released from distal to proximal, which means first the two distal ends of the implant, until finally also the most proximally located areas of the distal implant arms, which are still inside the sleeve, emerge from the distal sleeve sections. This can be achieved, for example, by increasing the force to be exerted in the proximal direction for opening the opening zone from distal to proximal. For example, the resistance of a weakening zone can become stronger from distal to proximal by increasing the material thickness, using different materials or making use of a perforation with intermediate segments that increase in thickness in the proximal direction and/or openings that increase in the distal direction. In the event a longitudinal slot is provided, the overlap of the edges of the longitudinal slot may increase from distal to proximal, which also results in the distal ends of the implant being released first. With the aid of the insertion system described, the individual implant arms can be released separately by retracting the sleeves or the trunk sleeve in the proximal direction. At least in the area of the distal sleeve sections, the sleeves are usefully made of a flexible tube material so that the opening zones can be opened easily thus allowing the implant to exit. Also the trunk sleeve can be made of a flexible tube material.

Another insertion system that can be employed is provided with two distal shaft sections that extend through the two distal implant arms, as well as a proximal shaft section that extends through the proximal implant arm. The three shaft sections are connected to each other adjacent to the branching point of the implant. Retraction of the distal shaft sections in the proximal direction can also be accomplished via the proximal shaft section. The distal implant arms are releasably secured to the distal shaft sections so that the distal implant arms can assume their expanded structure when securing is undone causing the arms to be released in the branching blood vessels.

With this insertion system, the implant is thus not placed in the desired position within a sleeve, but is secured on the shaft sections of the insertion system. In other words, the implant is arranged on the insertion system when moved to the desired target site. As soon as correct placement is achieved, the fixation of the implant to the shaft sections is released. The implant then expands and adapts to the inner vessel wall, with the two distal implant arms adapting to the inner vessel wall of the two branching blood vessels.

The fixation points securing the distal implant arms to the distal shaft sections are usefully located at least at the distal ends of the implant. As soon as the fixation points at the distal ends are unfastened, expansion of the implant begins. Additional fixation points arranged further proximally are also possible, but these are not essential because a release to occur further proximally can also be controlled via a microcatheter which surrounds the implant and the insertion system.

The shaft sections can in particular be made up of a flexible tubular material, which offers the advantage that the implant adheres well to the tubular material and an additional frictional connection is produced between the implant and the tubular material. However, it is also absolutely conceivable to use a metallic shaft, for example, on which the implant is mounted and secured.

Expediently, the shaft sections comprise an internal lumen because this allows one or more guidewires to pass through, via which the insertion system can be moved to the desired location. In particular, several guide wires can also be used, one of which, for example, is guided into the first branching vessel and the other into the second branching vessel in order to be able to advance the insertion system to the desired target site.

The fixation points may be provided in the form of connections designed to be detached chemically, thermally, electrolytically or mechanically. Detaching of the joints can be suitably controlled by appropriately setting the respective parameters.

Chemically, thermally or electrolytically detachable connection points are understood to be connections that can be dissolved by chemical or thermal action or electrolytically by applying an electrical voltage at least to such an extent that the respective arm of the implant detaches from the shaft section.

The connection points may be adhesive bonds, preferably created using a polymer adhesive. In this case, the implant is detached chemically, typically by applying a solvent that at least partially dissolves the adhesive. DMSO (dimethyl sulfoxide), for example, can be used as a solvent. A chemically detachable connection point shall also be understood as a junction that is detached by exposure to the surrounding blood alone.

Another possible method is the electrolytic detachment of the connection points. In this case, at least partial dissolution of the connection point is achieved by applying an electrical voltage. The electrolytic detachment of implants is well known practice in the state of the art. Suitable materials for the connections to be dissolved electrolytically include stainless steel, magnesium, magnesium alloys or cobalt-chromium alloys. A particularly preferred magnesium alloy is Resoloy®, which was developed by the company MeKo from Sarstedt/Germany (cf. WO 2013/024125 A1). It is an alloy consisting of magnesium and, inter alia, of lanthanides, in particular dysprosium. Another advantage of using magnesium and magnesium alloys is that magnesium residues remaining in the body are physiologically unproblematic.

In the event of thermally detachable connections, a heat source can be brought to act on the connection points to be detached in order to release the implant.

Another approach to securing the shaft sections with the implant arms intended for placement in the branching blood vessels is to place caps on the distal ends of the distal implant arms to prevent the implant arms from expanding radially. These caps can be removed in the distal direction from the distal ends, in particular can be pushed off, in order to achieve a release of the implant in this way.

In particular, the caps may be cylindrical in shape, and generally the distal ends of the caps are nearly entirely closed except for a small opening for passage of a wire as described further below. The inner diameter of the caps must be such that expansion and release of the distal implant arms are prevented when the caps are in place.

In particular, removal of the caps can in particular be brought about by passing push wires at least temporarily through the inner lumen of the distal shaft sections. These push wires are designed such that advancement of the push wires in the distal direction causes the caps to slide off the distal ends of the implant. For this purpose, the push wires may, for example, have thickenings arranged on or near the distal end.

Particularly preferred is the provision of push wires which extend through openings provided for this purpose in the caps, with the push wires having thickenings distal and proximal to the openings, and with the thickening of a push wire located further proximally ensuring that the thickening rests against the interior of the cap during advancement in the distal direction and pushing the cap away from the implant when further force is exerted, while the distal thickenings ensure that the push wires are held in their positions and do not slip out of the openings in the caps. The thickenings should have a diameter larger than the diameter of the openings in the caps provided for the push wires, as otherwise it would be possible for the push wires to become detached from the caps. In particular, the thickenings can be spherical in shape.

When using one of the insertion systems described or any other insertion system, the implant and insertion system can be brought to the target site via a microcatheter.

Aside from the implant itself, the invention also relates to the use of the implant for the treatment of arteriovenous malformations, in particular (bifurcation) aneurysms, as well as to combining the implant with an insertion system and, as the case may be, a microcatheter. All statements made with reference to the implant itself also apply in an analogous manner to the use of the implant and to a method of using the implant.

Further elucidation of the invention is provided by way of examples through the enclosed figures. It is to be noted that the figures show preferred embodiment variants of the invention, but with the invention itself not being limit thereto. To the extent it is technically expedient, the invention comprises, in particular, any optional combinations of the technical features that are stated in the claims or in the description as being relevant to the invention.

Clarification of the invention is provided by the following figures where

FIG. 1 shows a first braided section according to the first embodiment;

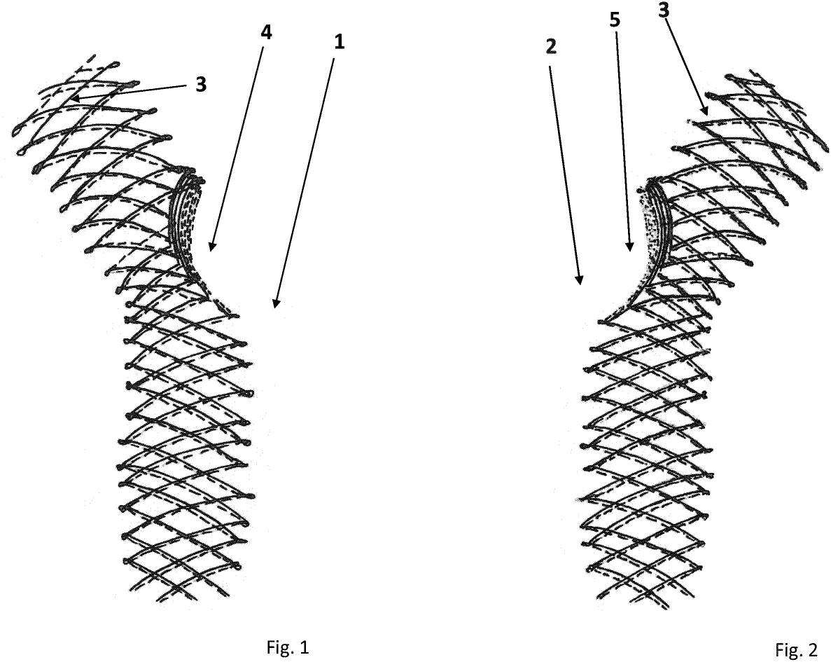

FIG. 2 shows a second braided section according to the first embodiment;

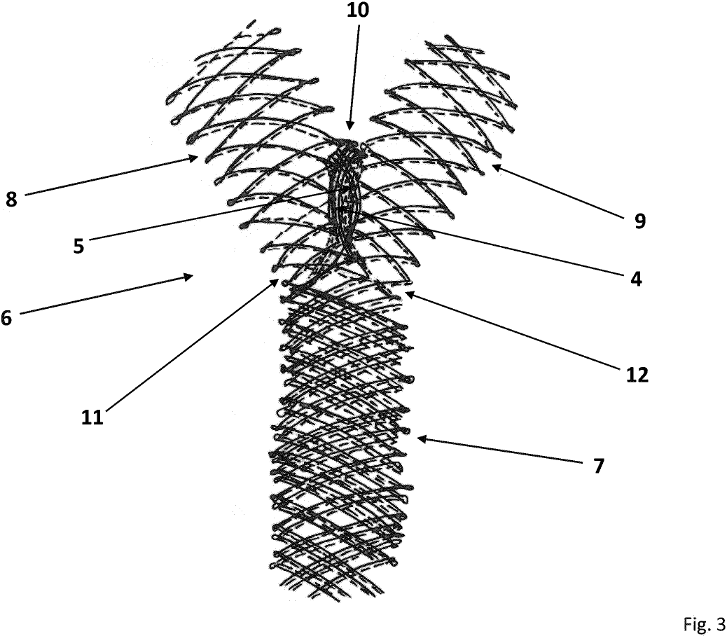

FIG. 3 illustrates the implant assembled from first and second braided sections according to the first embodiment;

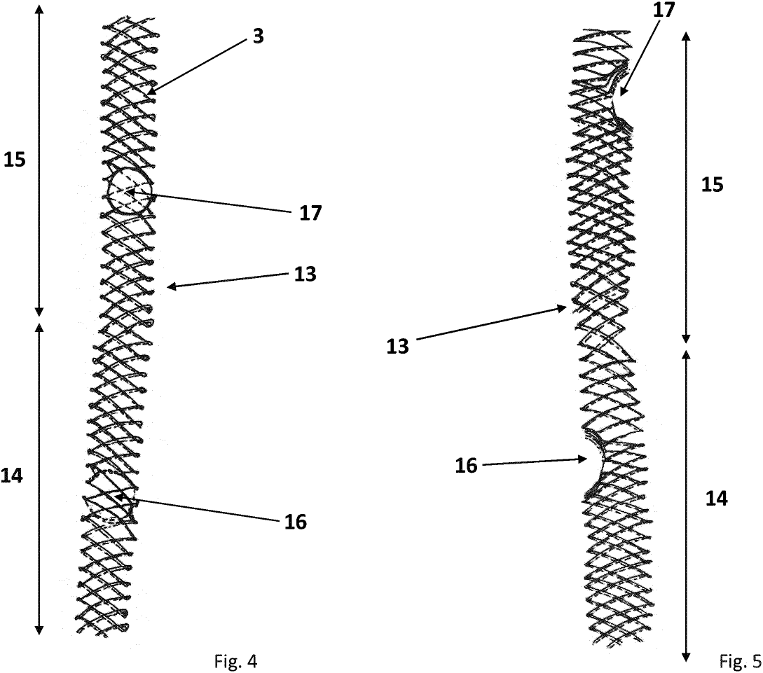

FIG. 4 is a plan view of a braided structure according to the second embodiment;

FIG. 5 is a side view of a braided structure according to the second embodiment; and

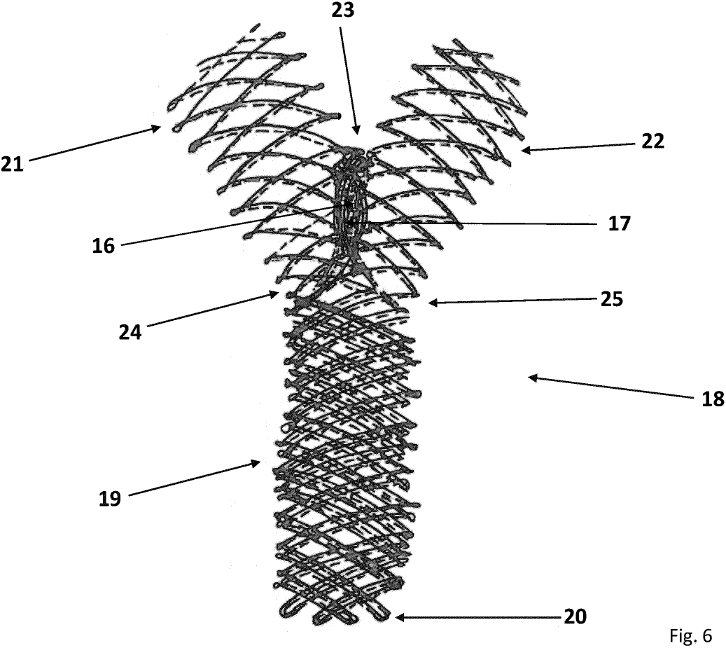

FIG. 6 shows the implant formed from the braided structure according to the second embodiment.

FIGS. 1 to 3 show the first embodiment of the invention. In FIG. 1 the first braided section 1 is shown according to the first embodiment of the invention. This section is made up of a plurality of wires 3 which are interwoven with each other. Wires 3 in the foreground are shown with a solid line, wires 3 in the background with a dashed line. Braided section 1, when expanded, forms a substantially tubular structure having an opening 4 in the wall, said opening being suitably sized so as to allow the other braided section 2 to be passed through the opening.

FIG. 2 corresponds to FIG. 1, but shows second braided section 2, which is of essentially identical configuration, i.e. also made of interwoven wires 3. This braided section 2 also has an opening of a size that enables the other braided section 1 to be passed through.

FIG. 3 is an illustration of the implant 6 assembled from the first and second braided sections 1, 2. Implant 6 has a Y-shape, with the two openings 4, 5 in the braided sections 1, 2 facing each other so that the flow of blood between arms 7, 8, 9 is not impeded. One of the arms 7 is of double-layered configuration, because the two braided sections 1, 2 are superimposed in this area. The other two arms 8, 9 are each single-layered because merely the first and second braided sections 1, 2 are located here. Accordingly, the surface coverage is greater in the double-layer arm 7 than in the two single-layer arms 8, 9.

The standard placement procedure of the implant 6 is such that area 10 comes to be located in front of the aneurysm, respectively in the aneurysm neck. This arrangement results in the double-layered arm 7 to be anchored in the parent blood vessel, while the two single-layered arms 8, 9 extend into the branching blood vessels. Blood is thus allowed to flow unimpeded from the parent blood vessel into the two branching blood vessels via openings 5, 6 facing each other in the braided sections 1, 2. Furthermore, the aneurysm is largely isolated from flow of blood because the braid density at position 10 is sufficiently high.

Optionally, the implant 6 may be placed so that either area 11 or area 12 comes to be located in front of the aneurysm. In this case, the double-layered arm 7 is positioned in one of the branching blood vessels, while one of the two single-layered arms 8, 9 being placed in the parent blood vessel. This can be advantageous in that the braid density in areas 11 and 12 is usually even higher than in area 10.

FIGS. 4 to 6 are illustrations of the second embodiment of the invention. In FIG. 4 the braided structure 13 is depicted, which comprises first and second braided sections 14, 15. However, unlike in the first embodiment, both braided sections 14, 15 form part of a single braided structure 13 and are arranged one after the other in the longitudinal direction. An opening 16, 17 can be seen in each of the two braided sections 14, 15, with, in the representation chosen here, opening 17 pointing upward and opening 16 pointing downward as shown. Accordingly, the openings 16, 17 are thus located on opposite sides. The braided structure 13 is as well composed of interwoven wires 3, wherein wires 3 located in the foreground are again shown with a solid line, and wires 3 located in the background are shown with a dashed line.

FIG. 5 is identical to the illustration in FIG. 4, but shown in a side view, with the opposite arrangement of the openings 16, 17 being clearly visible.

In FIG. 6, the implant 18 according to the second embodiment is shown in its entirety after it has been formed into the appropriate shape. To achieve this, the first braided section 14 was turned to the inside and passed through the braided structure 13 such that it was allowed to exit at the opening 17 in the second braided section 15. In this way, a Y-shaped implant 18 is again obtained overall with three arms 19, 21, 22, of which one arm 19 is double-layered, while the other two arms 21, 22 are single-layered. Unlike the first embodiment, however, the outer end 20 of the double-layered arm 19 does not exhibit free wire ends since the outer layer is connected to the inner layer due to the manufacturing method described. The absence of free wire ends is advantageous in that it makes the implant 18 less traumatic at this point so that the risk of injury to the vessel wall is reduced.

Also in the second embodiment, the two openings 16, 17 face each other so that blood flow through the parent blood vessel and the two branching blood vessels is unobstructed. On the other hand, however, the aneurysm in front of which implant 18 is positioned is effectively separated from the blood stream because implant 18 has a sufficiently high surface density at the position where it is located in front of the aneurysm neck. Following the standard positioning procedure, this is area 23, but also in this embodiment it is alternatively possible to place areas 24 or 25 in front of the aneurysm to achieve an even higher braid density immediately in front of the aneurysm neck. Whereas the standard positioning procedure calls for placing the double-layered arm 19 in the parent blood vessel, the two alternative methods of positioning require the double-layered arm 19 to be placed in one of the branching vessels. Accordingly, one of the two single-layer arms 21, 22 must then be placed in the parent blood vessel.

1. An implant for influencing the flow of blood in the area of aneurysms which are localized at vessel branchings, with the implant (6, 18) being provided in an expanded state in which it is implanted in the blood vessel and in a contracted state in which it is movable through the blood vessel, with the implant (6, 18) having a first (1, 14) and a second braided section (2, 15) which are tubular in the expanded state and the walls of which are composed of individual wires (3) interwoven with one another, the first (1, 14) and second braided sections (2, 15) each having an opening (4, 5, 16, 17) in the wall, with at least the size of the opening (5, 17) in the second braided section (2, 15) being sufficient for the passage of the first braided section (1, 14) wherein
the openings (4, 5, 16, 17) in the walls being produced in such a way that the wires (3) forming the braided sections (1, 2, 14, 15) are radially displaced at the positions of the openings (4, 5, 16, 17), and the first braided section (1, 14) is guided through the opening (5, 17) in the wall of the second braided section (2, 15) such that the opening (4, 16) in the wall of the first braided section (1, 14) points to the opening (5, 17) in the wall of the second braided section (2, 15).

2. An implant according to claim 1, wherein the number of wires (3) forming the respective braided sections (1, 2, 14, 15) is 24 to 96.

3. An implant according to claim 1, wherein in the expanded state, the first (1) and second braided sections (2) overlap in the segment (7) in which the two braided sections (1, 2) jointly extend, in such a way that the inner braided section (1) projects beyond the outer braided section (2).

4. An implant according to claim 1, wherein the first (14) and the second braided sections (15) are connected to each other at the outer end (20) of the segment (19) in the segment in which the two braided sections (14, 15) jointly extend.

5. An implant according to claim 1, wherein the first (1, 14) and second braided sections (2, 15) are attached to each other in the region of their openings (4, 5, 16, 17).

6. An implant according to claim 5, wherein the fixation in the area of the openings (4, 5, 16, 17) is performed by sewing and/or by loops guided around the wires (3).

7. An implant according to claim 1, wherein the openings (4, 5, 16, 17) in the walls of the first (1, 14) and/or second braided section (2, 15) are arranged approximately in the middle with respect to the longitudinal extension.

8. An implant according to claim 1, wherein the wires (3) are composed at least partially of a material having shape memory properties.

9. An implant according to claim 8, wherein the material having shape memory properties is a nickel-titanium alloy.

10. An implant according to claim 8, wherein the wires (3) comprise a core of a radiopaque material and a sheath of a material having shape memory properties.

11. An implant according to claim 1, wherein the implant (6, 18) is provided with radiopaque markers.

12. An implant according to claim 1, wherein the implant (6,18) is provided with one or several membranes at least partially covering the braided sections (1, 2, 14, 15).

13. An implant according to claim 12, wherein the membrane has an antithrombogenic effect or an effect that promotes endothelial formation.

14. An implant according to claim 1, wherein the number of wires (3) forming the respective braided sections (1, 2, 14, 15) is 36 to 64.
